(19) United States
(12) Patent Application Publication
(10) Pub. No.: US 2008/0249474 A1
(43) Pub. Date: Oct. 9, 2008

(54) INTRALUMINAL AND TRANSLUMINAL DEVICE AND METHOD OF VISUALIZATION AND THERAPEUTIC INTERVENTION

(75) Inventor: Randal S. Baker, Ada, MI (US)

Correspondence Address: VAN DYKE, GARDNER, LINN & BURKHART, LLP, SUITE 207, 2851 CHARLEVOIX DRIVE, S.E., GRAND RAPIDS, MI, 49546, US

(73) Assignee: SENTINEL GROUP, LLC, Grand Rapids, MI (US)

(21) Appl. No.: 12/117,363

(22) Filed: May 8, 2008

Related U.S. Application Data
(63) Continuation-in-part of application No. PCT/US06/60737, filed on Nov. 9, 2006.
(60) Provisional application No. 60/597,107, filed on Nov. 10, 2005.

(51) Int. Cl. A61B 17/00

(52) U.S. Cl. 604/167.02; 606/191

(57) ABSTRACT

A natural orifice luminal device and method of performing a natural orifice luminal procedure includes providing a generally closed body. The generally closed body has a size and shape to be retained by a portion of the lumen defining an aperture between the lumen and a space adjacent to the lumen. The generally closed body is adapted to substantially seal the aperture from passage of gas between the lumen and the space adjacent to the lumen. In this manner, the lumen and the space adjacent to the lumen can be at different gas pressures.

### CROSS REFERENCE TO RELATED APPLICATIONS

The present invention is a continuation-in-part application of International Application No. PCT/US2006/060737, filed on Nov. 9, 2006, which claims the benefit of U.S. provisional patent application Ser. No. 60/597,107, filed on Nov. 10, 2005, the disclosures of which are hereby incorporated herein by reference in their entireties.

### BACKGROUND OF THE INVENTION

The present invention is directed to a natural orifice luminal device and method of performing a natural orifice luminal procedure. The present invention is also directed to a natural orifice transluminal device and method of performing natural orifice transluminal endoscopic surgery. While the invention is illustrated for use in the stomach, it may be applied to the vagina, bladder, colon, and other natural orifices.

Various procedures require that a lumen, such as the stomach, be insufflated by a pressurizing gas, such as air or carbon dioxide. However, the pressuring gas tends to flow beyond the lumen into adjacent spaces, such as adjacent cavities, during certain procedures. For example, whenever the stomach is pressurized, the pressurizing gas tends to flow to the lower gastrointestinal (GI) track, causing it to become distended. Even when only a minor amount of gas passes to the lower GI track, discomfort to the patient may arise. The distended GI track may encroach on the organs of the abdominal cavity. In extreme cases, a condition known as abdominal compartment syndrome (ACS) may occur. ACS arises when distension of the bowel decreases the domain of the abdomen to the extent that it affects operation of other organs either directly or by decreasing venous return flow. This may affect the kidneys, the liver, the pancreas and the like. Also, intrahepatic portal venous gas and pneumatosis cystoides intestinalis may occur as a result of the air entering the portal system that drains the liver system resulting in potential harm to the biliopancreatic functioning. In the case of upper GI bleeding, swelling of the bowel from the insufflation gas may cause the surgeon to discontinue an attempt to get the vessel to stop bleeding endoscopically. This could result in a necessity to continue the procedure with a more invasive procedure in an operating room.

A pressure differential also may occur between a lumen, such as the stomach, and a surrounding space, such as the peritoneal cavity, during natural orifice transluminal endoscopic surgery (NOTES). During NOTES, the surgeon makes an incision in the lumen wall and passes an instrument, such as an endoscope, through the stomach wall into the peritoneal cavity. The peritoneal cavity may be pressurized above atmospheric pressure with a gas, such as air or carbon dioxide, to distend the walls thereof to improve visualization of the organs and therapeutic intervention.

### SUMMARY OF THE INVENTION

The present invention is directed to providing a natural orifice luminal device and method and transluminal device and method to enhance the performance of natural orifice luminal procedures and transluminal endoscopic surgery and procedures.

A natural orifice luminal device and method of performing a natural orifice luminal procedure, according to an aspect of the invention, includes providing a generally closed body. The generally closed body has a size and shape to be retained by a portion of the lumen defining an aperture between the lumen and a space adjacent to the lumen. The generally closed body is adapted to substantially seal the aperture from passage of gas between the lumen and the space adjacent to the lumen. In this manner, the lumen and the space adjacent to the lumen can be at different gas pressures.

The generally closed body can have a size and shape to be substantially entirely within the lumen and the space adjacent to the lumen upon deployment. The generally closed body may be adapted to be compressed during deployment and expanded to at least the size of the aperture between the lumen and the space adjacent to the lumen upon completion of deployment. The generally closed body may be made substantially from a self-expanding material.

The generally closed body may include a diaphragm and one or more retention flange(s) generally surrounding the diaphragm. The retention flange is adapted to retain the generally closed body in the aperture between the lumen and the space adjacent to the lumen. The diaphragm may be generally impenetrable. The generally closed body may include a handle for manipulating the generally closed body. The generally closed body may be configured to be retained by the stomach wall at the pylorus or by the stoma of a gastric pouch.

The diaphragm may be penetrable. The body may include a self-closing through-opening in the diaphragm. The self-closing through-opening may include a mechanical seal or a pneumatic seal. The generally closed body may be configured to be retained by the stomach wall at the pylorus or by the stoma of a gastric pouch. The generally closed body may be configured to be retained by a portion of the lumen wall at an incision in the lumen wall between the lumen and the peritoneal cavity or abdominal cavity.

A natural orifice transluminal device and method for performing natural orifice transluminal endoscopic surgery, according to an aspect of the invention, includes providing a device having a body with a size and shape to be supportable by a portion of a luminal wall surrounding an incision. Instruments are passed through the incision while protecting, with the body, the wall of the lumen at the incision from abrasion resulting from the passage of instruments. The luminal wall is sealed at the incision with the body against the exchange of fluid between the lumen and the space adjacent to the lumen. The device is adapted to facilitate closing of the incision after the surgery.

The body may have a size and shape to be substantially within the lumen and the space adjacent to the lumen upon deployment. The body may be deployed in the incision with a trans-orifice instrument. The body may be made at least in part from a self-expanding material.

The body may define a diaphragm and one or more retention flange(s) generally surrounding the diaphragm. The at least one retention flange may support the body in the aperture in the wall of the lumen. An opening may be defined in the diaphragm and a seal provided to allow the passage of instruments through the opening while substantially sealing the opening against the exchange of fluid. The seal may be a mechanical seal and/or a pneumatic seal.

The device may be made at least in part from a bioabsorbable material. The device may be made from a plurality of bioabsorbable materials, each adapted to absorb in the patient at a different rate. This allows the incision to remain sealed while the size of the body is reduced as the incision heals. The device may include a cap to cover the body after the natural orifice transluminal surgery. The cap may be made from a bioabsorbable material that absorbs in the patient at a slower rate than the bioabsorbable material making up the body.

A closing mechanism may be provided to close the incision in the luminal wall. The closing mechanism may be configured to be passed through the body in order to close the incision in the luminal wall from the direction of the space adjacent the luminal wall. The closing mechanism may include a plurality of tines directable toward the lumen wall. The tines may be biased away from each other and compressible toward each other to pass through the body. A closing mechanism deployment tool may be provided to compress the tines during deployment. The closing mechanism may be made at least in part from a material that is bioabsorbable.

A device deployment tool may be provided for deploying the device to the luminal wall. The device deployment tool may include a tube for compressing the device to a size that passes through the natural orifice and lumen. The device deployment tool may include a lance and/or a needle to form an incision in the lumen wall and a dilator to expand the incision in the lumen wall. A passage may be defined through the lance, the needle or the dilator. The passage may be used to insufflate the space adjacent to the lumen. The lance, the needle and/or the dilator may be withdrawn through the penetrable diaphragm after deployment of the device.

A natural orifice luminal device and method of performing a natural orifice luminal procedure, according to another aspect of the invention, includes providing a generally closed body. The generally closed body has a size and shape to be retained by a portion of the lumen. The generally closed body substantially seals the lumen from passage of gas used to insufflate the lumen to a space distal the lumen with respect to the natural orifice.

The generally closed body may be adapted to be compressed during deployment through the natural orifice and expanded to at least the size of the portion of the lumen upon completion of deployment. The generally closed body is made substantially from a self-expanding material.

The generally closed body may be configured to be deployed transorally and to be retained at the pylorus. In this manner, the passage of gas used to insufflate the stomach is generally blocked from the bowel.

The generally closed body may be deployed transorally and retained at the stoma of a gastric pouch. In this manner, passage of gas used to insufflate the pouch is generally blocked from the bowel.

The generally closed body may include a diaphragm and at least one retention flange generally surrounding the diaphragm. The retention flange retains the generally closed body at the portion of the lumen. The diaphragm may be generally impenetrable or penetrable. A self-closing through-opening may be provided in the diaphragm. The generally closed body may include a handle that may be used to manipulate the generally closed body.

The natural orifice luminal device may be used in combination with another device having a body with a size and shape to be supported in an incision in the luminal wall. The another body is penetrable to pass instruments through the incision while protecting the wall of the lumen at the incision from abrasion resulting from the passage of instruments. The another body may substantially seal the luminal wall at the incision against the exchange of fluid between the lumen and the space adjacent to the lumen.

These and other objects, advantages and features of this invention will become apparent upon review of the following specification in conjunction with the drawings.

### BRIEF DESCRIPTION OF THE DRAWINGS

FIG. 1 is a sectional view of a lumen, such as the stomach, illustrating a natural orifice luminal device, according to an aspect of the invention, positioned at the pylorus and a natural orifice transluminal endoscopic device, according to the an aspect of the invention, positioned in an incision in the stomach wall with an endoscope passing through the latter;

FIG. 2 is a perspective view of a natural orifice luminal device, according to an aspect of the invention, having an impenetrable diaphragm;

FIG. 3 is the same view as FIG. 2 of a natural orifice luminal device, according to an aspect of the invention, having a penetrable diaphragm;

FIG. 4 is a side elevation of the luminal device in FIG. 3 with a surgical tool 120 passing through the penetrable diaphragm;

FIG. 4a is the same view as, FIG. 4 of an alternative embodiment thereof;

FIG. 5 is a perspective view of a device deployment tool;

FIG. 6 is a side elevation of a patient's stomach illustrating one technique for closing an incision in the lumen wall;

FIG. 7 is a side elevation of a closing mechanism for closing the incision in the stomach wall;

FIG. 8 is a perspective view of a closing mechanism deployment device for use with the closing mechanism of FIG. 7;

FIG. 9 is the same view as FIG. 1 of an alternative embodiment;

FIG. 10 is a top plan view of the natural orifice luminal device illustrated in FIG. 9;

FIG. 11 is a sectional view taken along the lines XI-XI in FIG. 10; and

FIG. 12 is a sectional view illustrating a technique for deployment of the natural orifice luminal device illustrated in FIGS. 9-11.

### DETAILED DESCRIPTION OF THE PREFERRED EMBODIMENT

Referring now to the drawings and the illustrative embodiments depicted therein, a natural orifice luminal device and method includes providing a device 10, 110 having a respective generally closed body 11, 111 that is adapted to be retained by a portion of a lumen, such as the stomach, defining an aperture. Closed body 11, 111 includes a diaphragm 14, 114, a first retention flange 12, 112, generally surrounding the respective diaphragm and a second retention flange 13, 113 spaced apart from the other retention flange. Diaphragm 15, 115 which includes a cylindrical wall 14, 114, respectively, generally seals the opening or aperture between the lumen and the space adjacent to the lumnen. For natural orifice luminal device 10, the cylindrical wall 14 is illustrated as retained by the portion of the wall of the stomach at the pylorus to thereby maintain a pressure differential between the stomach and the intestines to reduce the flow of air into the bowel distally. This allows the surgeon to insufflate the stomach in order to inspect the mucosa, control upper GI (gastrointestinal) bleeding, or the like, without distending the intestines. If the procedure is to be entirely within the stomach, diaphragm 15 may be generally impenetrable.

Natural orifice luminal device 110 includes a generally closed body 111 having a penetrable diaphragm 115. The penetrable diaphragm may define a self-closing through-opening 22 thereby allowing diaphragm 115 to be penetrable, such as by an endoscope 20 or other transluminal tool, such as surgical tool 120. Also, multiple transluminal endoscopic tools may pass through the luminal wall by the use of multiple closed bodies 111. However, other techniques may be used. Device 110 is illustrated positioned at an incision in the stomach wall with cylindrical wall portion 114 sealing the incision in the stomach wall to substantially prevent flow of gas or liquids between the stomach and the peritoneal cavity. Retention flanges 112, 113 span the lumen wall to thereby provide self-supporting retention of body 111 at the portion of the luminal wall surrounding the incision. This penetrable sealing of the luminal wall allows the surgeon to maintain different gases and/or pressures in the lumen, such as the stomach, and the space adjacent to the lumen, such as the peritoneal cavity, while resisting infection of the peritoneal cavity from enzyme and other fluid leakage. Prior positioning of device 10 in the pylorus allows the surgeon to insufflate the stomach, such as with air or carbon dioxide, in order to perform the procedure to deploy the device 110 in the stomach wall while reducing the flow of air distally. Device 110 allows the surgeon to insufflate the peritoneal cavity, such as with air or carbon dioxide, either at the same pressure as the stomach or at a different pressure as will be described in more detail below. If the same pressure is to exist at the stomach and the peritoneal cavity, two different gases may be used without intermixing. If two different pressures are to be maintained in the stomach and the peritoneal cavity, the different pressures may be maintainable by the use of luminal device 110.

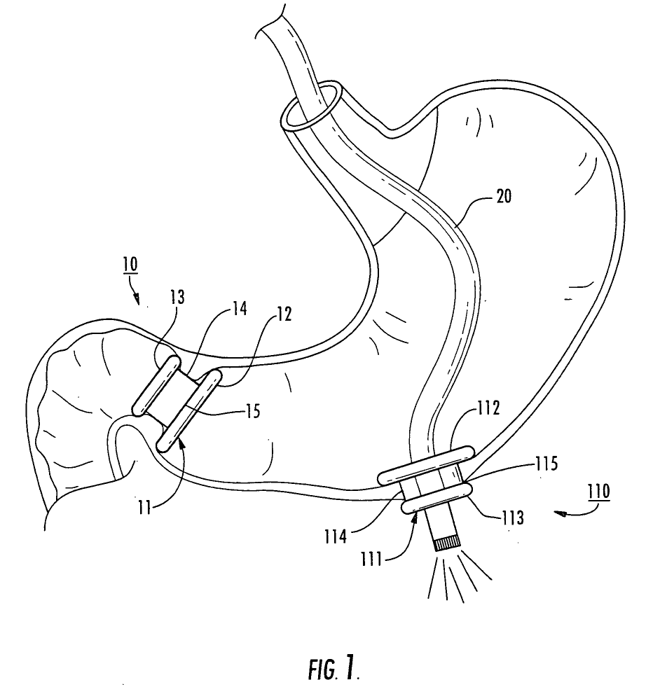

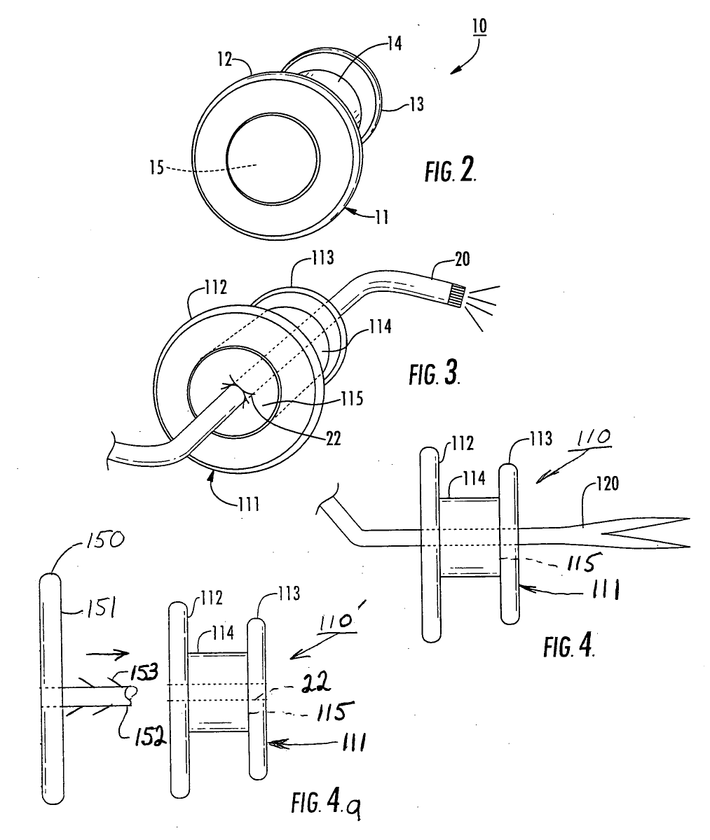

The use of a self-closing opening 22 allows the insertion of a natural orifice transluminal endoscopic tool 20, 120, such as an endoscopy, for visualization or a therapeutic device, such as a surgical instrument, through the opening into the space adjacent the lumen while preventing gas passage between the lumen and the space adjacent the lumen. It should be apparent to the skilled artisan that luminal devices 10, 110 may be used together as illustrated in FIG. 1 or separately. Also, luminal device 110 having a penetrable diaphragm may be positioned in the pylorus in order to reduce the flow of insufflating gas into the bowel distally while allowing the surgeon to access the duodenum or other portion of the bowel for visualization and/or therapeutic procedure as a part of an inspection of the stomach and/or procedure to control of bleeding in the upper GI track. Self-closing opening 22 may be a mechanical seal, such as a flapper valve, trapdoor, duckbill valve, or by the use of a self-closing penetrable material. Alternatively, self-closing opening may be a pneumatic seal, such as an air bridge.

Generally closed body 11, 111 may be made of a bioabsorbable material or a non-absorbable material. Generally closed body 11, 111 may be made from a synthetic or bioprosthetic material Examples of such materials include ePTFE, silicone, or the like. Body 11, 111 may be made with a coated mesh wall made from metal or carbon fibers, a coated coil configuration, a coated laser cut Nitinol sheet, a self-expanding polymer, or the like. If non-absorbable, the body 11, 111 is removed by the surgeon following the procedure. With luminal device 110, if body 111 is made of a bioabsorbable material, it may be left in the incision and absorbed into the patient while the incision is allowed to re-approximate or heal. The body seals the incision against the passage of fluid while facilitating closure of the incision by healing as the device body is absorbed in the patient.

In order to deploy device 10, 110, the generally closed body 11, 111 may be compressible to a size that allows the body to be passed through the natural orifice and lumen, such as the mouth and esophagus. Once in position, the body is allowed to expand to a deployment position. This may be accomplished by manufacturing the body from a self-expanding material, which may include a separate member within the body that facilitates the self-expansion of the generally closed body. Alternatively, the generally closed body may be expanded by a mechanism (not shown), such as a balloon within the body. In addition to being made of a pliable, compressible material, device 10, 110 may be made from one or more rigid or semi-rigid components that are hinged in a way that they may be compressed in order to be passed through the esophagus. In addition to being deployed trans-orally, in the illustrated embodiment, device 10, 110 may be deployed through the colon. If deployed through the colon, it may be deployed with a retrieval line attached to allow the device to be retrieved without inserting a retrieval instrument. As previously set forth, the techniques disclosed herein may be applied to other natural orifices and lumens, such as the vaginal, bladder, biliary duct, and the like.

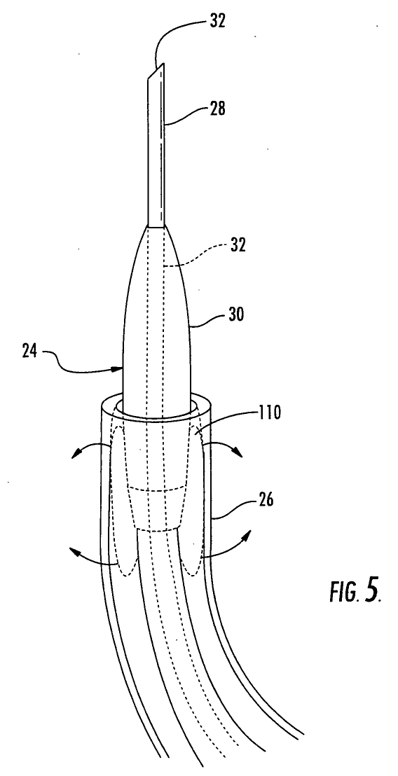

In order to deploy a device 110 in an incision in the luminal wall, such as the wall between the stomach and peritoneal cavity, the lumen is insufflated with air or carbon dioxide through a conventional over-tube, with a proximal seal of the over-tube maintaining the pressure in the lumen. A device deployment tool 24 may be fed through the lumen through the over-tube and an incision site in the lumen wall located (FIG. 5). Only the distal end of deployment tool 24 is illustrated in FIG. 5. Deployment tool 24 extends proximally out of the patient's natural orifice, such as the mouth, either directly or by being formed with another trans-oral instrument, such as an endoscope. Deployment tool 24 may include a tube 26 to compress device 110 sufficiently to pass through the natural orifice and proximal lumen, such as the esophagus. Tool 24 may additionally include a lance or needle 28 to form an initial incision in the lumen wall and dilator 30 in order to dilate the incision in the lumen wall sufficiently to deploy device 110. An insufflation passage 32 may extend through dilator 30 and/or lance/needle 28 in order to allow the surgeon to insuffiate the space adjacent the lumen, such as the peritoneal cavity, once the lance or needle passes through the lumen wall. The remainder of device deployment tool 24 may then be passed through the lumen wall until device 110 is juxtaposed with the incision in the lumen wall. Tube 26 may then be retracted using known techniques in order to allow gastric device 110 to self-expand into the incision in the lumen wall where the device will be self-supported, namely supported essentially entirely by the lumen wall, as illustrated by the arrows in FIG. 5. Once device 110 is deployed, dilator 30 and lance/needle 28 may be withdrawn as a unit from the opening 22 in diaphragm 115. Device 110 is now deployed in the lumen wall and allows a pressure differential to be maintained between the lumen and the area adjacent the lumen, such as between the stomach and the peritoneal cavity.

Deployment tool 24 may be guided to a potential incision site in the lumen using various techniques. One such technique involves trans-illumination. In trans-illumination, the surgeon positions an endoscope in the lumen, using known techniques, and turns the illumination setting to a high level. After the lights in the operating room are dimmed, the surgeon looks externally at the abdomen for the light source through the wall of the lumen and the skin. This allows the surgeon to locate an incision site where, for example, the bowel and other organs are not present. Of course, the bright light source could be external the patient with the endoscope searching for the light from within the lumen. Other techniques for locating an incision site could use other sensors to sense organs.

Once an incision site is identified, the surgeon guides deployment tool 24 to the incision site. The incision tool may be guided through the lumen using a blunt wire to avoid damage to the lumen as is known for guiding an endoscope. Once in the lumen, the deployment tool is guided to the incision site. This may be done using the tip of the endoscope to steer the distal end of the deployment tool. Alternatively, steering wires may be attached to the distal end of the deployment tool and terminated at a location external the patient. The steering wires can then be manipulated by the surgeon, while visualizing the lumen wall through an endoscope, until the deployment tool is at the desired incision site. Device deployment tool 24 may, alternatively, be positioned utilizing an optional parallel open working channel that is included with some endoscopes. Also, radiopaque markers may be applied to the device 10, 110 or to the device deployment tool. The deployment tool may then be manipulated within the lumen with fluoroscopic assist. Alternatively, the device deployment tool 24 may be guided utilizing a wire passed through the abdominal wall and into the stomach utilizing the technique disclosed in U.S. Pat. No. 6,918,871 B2 entitled METHOD FOR ACCESSING CAVITY, the disclosure of which is hereby incorporated herein by reference.

Once the device 110 is positioned at the incision in the luminal wall, the surgeon may perform known transluminal visualization and/or therapeutic procedures, such as utilizing an endoscope 20, a surgical tool 120, and the like, passing through opening 115 while reducing passage of gas and other fluids between the lumen and the space adjacent to the lumen. Upon completion of the transluminal procedure, the incision in the lumen wall is sealed while it is allowed to heal. This may be accomplished by manufacturing the generally closed body 111 of device 110 at least in part from a bioabsorbable material and left in the patient post-procedure, thereby sealing the incision but allowing the incision to heal while the gastric device is absorbed by the patient. This may bed further assisted by the placement of radiopaque markers in the gastric device. This allows monitoring of healing of the incision using x-ray imaging.

In another embodiment, a natural orifice luminal device 110′ includes a generally closed body 111 having a penetrable diaphragm 115 positioned in an incision in the lumen by a cylindrical wall portion 114 and one or more retention flanges 112, 113. The flanges span the lumen wall to provide self-supporting retention of body 111. Device 110′ additionally includes a cap 150 for sealing the opening 22 in diaphragm 115 left by the withdrawal of transluminal tool 120. Cap 150 is illustrated as having a sealing surface 151 and a support 152. Support 152 is configured to be inserted into opening 22 and sealing surface 151 is configured to seal opening 22. Of course the skilled artisan would understand that cap 150 may be configured in many ways to perform this function. Support 152 may include one or more barbs 153 to assist in retaining the cap into engagement with body 111.

Cap 150 may be made at least in part from a bioabsorbable material. In this manner, cap 150 may be absorbed in the patient along with generally closed body 111. Cap 150 may be made at least in part from a bioabsorbable material that has a different, slower, absorption rate than the bioabsorbable material used in body 111. This would allow cap 150 to remain in place while body 111 is absorbed as the incision heals. This allows the cap to continue to seal the incision against transfer of fluids between the lumen and the area surrounding the lumen until the incision is substantially closed. Alternatively, cap 150 could be inserted directly in the incision, such as if a non-absorbable material is used for body 111, after body 111 is removed post-surgically. The cap would seal the incision directly with sealing surface 151 while support 152 is retained by the wall of the lumen surrounding the incision. Cap 150 could be absorbed as the incision heals.

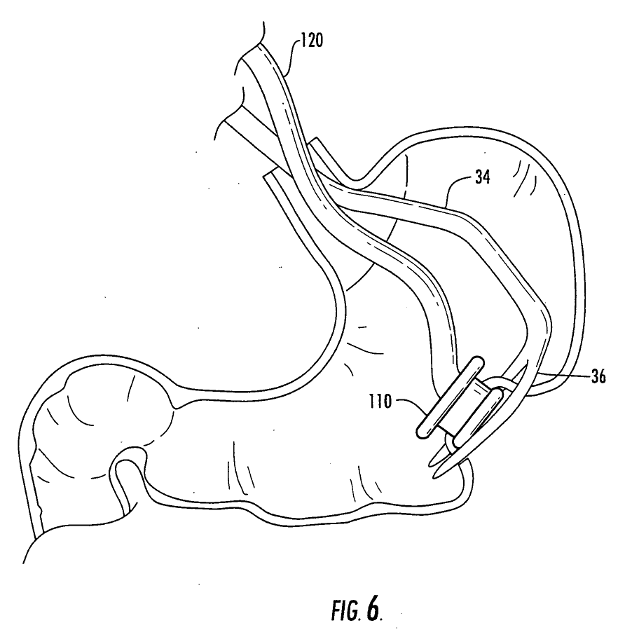

Alternatively, an incision closing mechanism 34 in the form of a natural orifice deployed clamp 36 and deployment handle may be used to approximate the lumen wall at the site of the incision (FIG. 6). This may be accomplished by the surgeon grasping device 110 with an endoscopic instrument and pulling the device proximally in order to pucker the lumen wall. The incision closing mechanism 34 may then be inserted endoscopically and the clamp 36 used to clamp the lumen wall distally of device 110. The deployment handle of incision closing mechanism 34 and luminal device 110 may then be removed from the incision site, which is now approximated for healing. Clamp 36 may be made from a bioabsorbable material to thereby be absorbed into the patient without further intervention by the surgeon. Alternatively, clamp 36 may be made from a non-absorbable material and subsequently removed by the surgeon in a post-operative endoscopy.

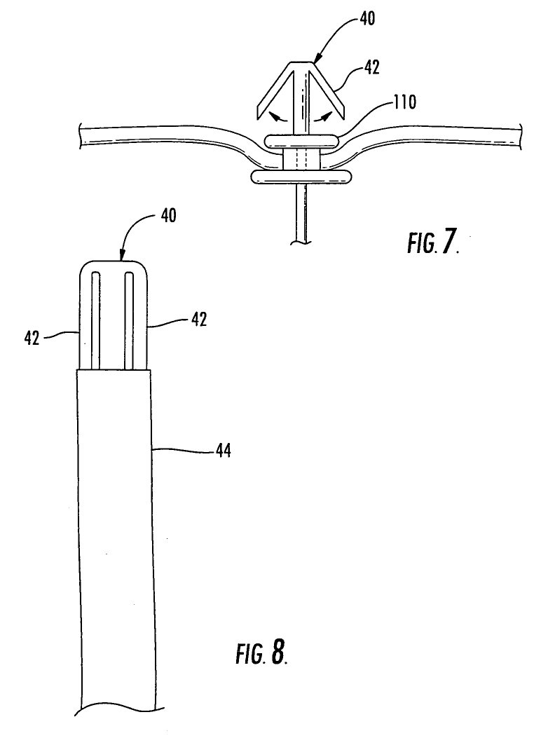

Alternatively, an incision closing mechanism 40 may be provided that is adapted to be deployed through the opening 22 in diaphragm 115 (FIG. 7). Closing mechanism 40 includes a plurality of tines 42 that may be biased away from each other. In order to pass closing mechanism 40 through opening 22 of the diaphragm upon completion of the transluminal procedure, tines 42 are compressed toward each other and are allowed to expand upon entering the cavity adjacent the lumen, such as the peritoneal cavity. The surgeon then retracts incision closing mechanism 40 proximally thereby inserting the tines 42 in the lumen wall surrounding the incision site. Once the tines are at least partially through the lumen wall, device 110 may be removed from the incision site and the tines 42 passed entirely through the lumen wall. A clamp, band, or other device may optionally be placed around the ends of tines 42 in order to retain the closing device in position and bring the tines closer together in order to approximate the incision site for healing. A closing mechanism deployment tool 44 may be provided in order to compress the tines 42 to a position sufficient to pass through opening 22 in diaphragm 115 (FIG. 8). Upon passage of the tines through the gastric device 110, closing mechanism deployment tool 44 may then be withdrawn in order to allow the tines 42 to self-deploy to an expanded position as illustrated in FIG. 7. Incision closing device 40 may be made of a bioabsorbable material such that tines 42 may absorb into the patient as the incision heals.

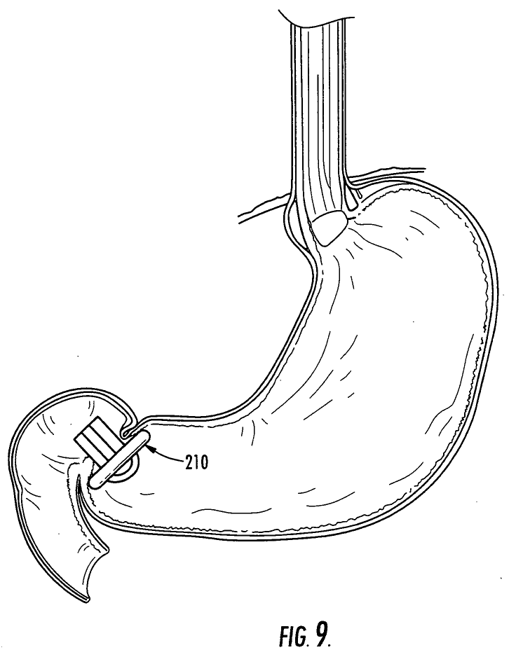

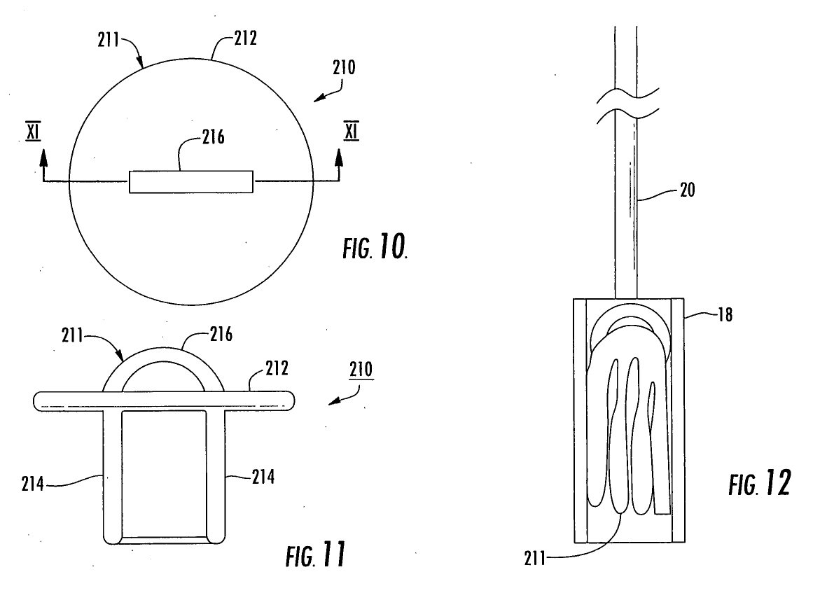

In an alternative embodiment, a luminal device, such as a plug, 210 includes a generally closed body 211 that is configured to the size and shape of an aperture between a lumen and the space adjacent to the lumen. In the illustrated embodiment, luminal device 210 is configured to seal the pylorus from the duodenum (FIGS. 9-12). However, luminal device 210 may be configured to other orifice/conduit combinations. For example, plug 210 may be configured to the size and shape of the stoma of a gastric pouch formed during gastric bypass surgery. This allows the surgeon to insufflate the pouch for a luminal procedure in the pouch without the gas distending the intestine. Luminal device 210 may include a generally circular retention flange 212 which seals against the aperture between he lumen and the space adjacent to the lumen to provide a generally gas-tight seal in the lumen. Luminal device 210 may additionally include a positioning portion, such as a generally cylindrical positioning wall 214 that stabilizes the device in the luminal apparatus, or the like. The function of luminal device 210 is to allow the lumen, such as the stomach or pouch, to be insufflated while the insufflation gas is impeded from passing distally of the natural orifice of the lumen, such as to the intestines. This allows the surgeon to visualize the lumen or to perform natural orifice endoscopic procedures, such as control of upper gastro-intestinal bleeding, without distending the space distal the lumen with the insufflation gas. For example, this reduces the likelihood of abdominal compartment syndrome (ACS) from distension of the bowel. This also reduces a decrease in the domain of the abdomen and its affect on the operation of the organs therein directly or by decreasing venous return flow. This is further useful in performance of natural orifice transluminal endoscopic surgery, such as illustrated in FIG. 1, because the domain of the peritoneal cavity is not reduced from distension of the bowels. It also may facilitate a more rapid recovery of the patient.

Device 210 may additionally include a structural feature, such as a handle 16, which allows the device to be engaged by an instrument, such as an endoscope. This allows the surgeon to readily remove the device at the end of the surgical or endoscopic procedure. Luminal device 210 may be made from various ones of the materials discussed above. Also, gastric device 210 may incorporate an expandable coil or other structure to make the generally closed body self-expanding. These materials allow the device to conform to the proximal portion of the lumen as it is being deployed through the lumen. Device 210 may be positioned within an insertion tube 18. Insertion tube 18, which may be manipulated by an instrument, such as an endoscope 20, compresses the device while moving through the lumen, such as through a conventional over-tube. Once at the deployment site, the device is deployed from tube 18 using conventional delivery techniques.

The various embodiments disclosed herein are especially useful with various natural orifice luminal procedures. This includes, by way of example, an esophageal gastro-duodenoscopy (EGD), control of upper GI bleeding, and the like. The embodiments may also be used with natural orifice transluminal endoscopic surgery, such as endoscopically assisted transgastric surgery, or the like. The embodiments facilitate visualization and therapeutic procedures by allowing the lumen, such as the stomach and the peritoneal cavity to be raised to pressure above atmospheric while reducing distal distension of the bowel and the attendant complications, including patient discomfort, abdominal compartment syndrome, pneumatosis, and the like. As previously set forth, the embodiments may be used with other orifice/conduit combinations.

Other applications for a device disclosed herein may be in other portions of the gastrointestinal tract. For example, a device may be placed proximally of a therapeutic procedure site in the large bowel to allow the bowel to be inflated. Another device may be placed distally of the site, such as therapeutic procedure devices to be passed through the distal device while more thoroughly sealing the site. While conditions, such as abdominal compartment syndrome, are not typically an issue during colon rectal procedures, the ability to inflate the bowel safely and effectively is enhanced.

Changes and modifications in the specifically described embodiments can be carried out without departing from the principles of the invention which is intended to be limited only by the scope of the appended claims, as interpreted according to the principles of patent law including the doctrine of equivalents.

The embodiments of the invention in which an exclusive property or privilege is claimed are defined as follows:

1. A natural orifice luminal device, comprising:
a generally closed body, said generally closed body having a size and shape to be retained by a portion of the lumen defining an aperture between the lumen and a space adjacent to the lumen, said generally closed body adapted to substantially seal the aperture from passage of gas between the lumen and the space adjacent to the lumen, wherein the lumen and the space adjacent to the lumen can be at different gas pressures.

2. The device as claimed in claim 1 wherein said generally closed body having a size and shape to be supported by the portion of the lumen defining the aperture between the lumen and the space adjacent to the lumen upon deployment.

3. The device as claimed in claim 1 wherein said generally closed body is adapted to be compressed during deployment and expanded to at least the size of the aperture between the lumen and the space adjacent to the lumen upon completion of deployment.

4. The device as claimed in claim 3 wherein said generally closed body is made substantially from a self-expanding material.

5. The device as claimed in claim 1 wherein said generally closed body comprises a diaphragm and at least one retention flange generally surrounding said diaphragm, said retention flange adapted to retain said generally closed body in the aperture between the lumen and the space adjacent to the lumen.

6. The device as claimed in claim 5 wherein said at least one retention flange comprises at least two closely spaced apart retention flanges.

7. The device as claimed in claim 5 wherein said diaphragm is generally impenetrable.

8. The device as claimed in claim 7 wherein said generally closed body includes a handle, said handle adapted to manipulate said generally closed body.

9. The device as claimed in claim 7 wherein said generally closed body is configured to be retained by the stomach wall at the pylorus or by the stoma of a gastric pouch.

10. The device as claimed in claim 5 wherein said diaphragm is penetrable.

11. The device as claimed in claim 10 including a self-closing through-opening in said diaphragm.

12. The device as claimed in claim 11 wherein said self-closing through-opening comprises a mechanical seal or a pneumatic seal.

13. The device as claimed in claim 10 wherein said generally closed body is configured to be retained by the stomach wall at the pylorus or by the stoma of a gastric pouch.

14. The device as claimed in claim 10 wherein said generally closed body is configured to be retained by a portion of the lumen wall at an incision in the lumen wall between the lumen and the space adjacent to the lumen.

15. A natural orifice transluminal device for use with a natural orifice transluminal endoscopic procedure, said device comprising:
a body having a size and shape to be supported by a portion of a luminal wall surrounding an incision;
wherein said body is penetrable to pass instruments through the incision while protecting the wall of the lumen at the incision from abrasion resulting from the passage of instruments while substantially sealing the luminal wall at the incision against the exchange of fluid between the lumen and the space adjacent to the lumen; and
wherein said device is adapted to facilitate closing of the incision after the surgery.

16. The device as claimed in claim 15 wherein said device further comprises a closing mechanism, said closing mechanism adapted to close the incision in the lumen wall.

17. The device as claimed in claim 16 wherein said closing mechanism is adapted to pass through said penetrable body and to close the incision in the lumen wall from the direction of the peritoneal cavity.

18. The device as claimed in claim 17 wherein said closing mechanism comprises a plurality of tines directable toward the lumen wall.

19. The device as claimed in claim 18 wherein said tines are biased away from each other and are compressible toward-each other to pass through said penetrable diaphragm.

20. The device as claimed in claim 17 including a closing mechanism deployment tool, said closing mechanism deployment tool adapted to compress said closing mechanism during deployment.

21. The device as claimed in claim 17 wherein said closing mechanism is made at least in part from a bioabsorbable material.

22. The device as claimed in claim 15 including a cap, said cap adapted to seal the incision against passage of fluid and wherein said body is adapted to be removed after surgery.

23. A natural orifice transluminal device for use with a natural orifice transluminal endoscopic procedure, said device comprising:
a body having a size and shape to be supported by a portion of a luminal wall surrounding an incision;
wherein said body is penetrable to pass instruments through the incision while protecting the wall of the lumen at the incision from abrasion resulting from the passage of instruments while substantially sealing the luminal wall at the incision against the exchange of fluid between the lumen and the space adjacent to the lumen; and
wherein said body is made at least in part from a bioabsorbable material, wherein said body seals the incision against the passage of fluid while facilitating closing of the incision by healing due to absorption of said body.

24. The device as claimed in claim 23 wherein said cap is made from a bioabsorbable material.

25. The device as claimed in claim 23 wherein said device is made from a plurality of bioabsorbable materials, each adapted to absorb in the patient at a different rate, wherein one of said materials absorbs at a slower rate thereby sealing the incision against the passage of fluid while another of said materials absorbs at a higher rate thereby facilitating closing of the incision by healing due to absorption of said another of said materials.

26. The device as claimed in claim 25 wherein said device includes a cap that is adapted to seal said body, wherein said cap is made from said one of said materials and said body is made from said another of said materials.

27. The device as claimed in claim 23 including a device deployment tool for deploying said device at the portion of the lumen wall.

28. The device as claimed in claim 27 wherein said device deployment tool comprises a tube that is adapted to compress said device to a size that passes through the lumen.

29. The device as claimed in claim 28 wherein said device deployment tool comprises at least one chosen from a lance and needle to form an incision in the stomach wall and a dilator to expand the incision in the lumen wall.

30. The device as claimed in claim 29 including a passage defined through said lance, said needle or said dilator, said passage adapted to insufflate the space adjacent to said lumen.

31. The device as claimed in claim 30 wherein said lance, said needle or said dilator are adapted to be withdrawn through said penetrable diaphragm after deployment of said device.

32. A natural orifice luminal device, comprising:
a generally closed body, said generally closed body having a size and shape to be retained by a portion of the lumen, said generally closed body adapted to substantially seal the lumen from passage of gas used to insufflate the lumen from the lumen to a space distal the lumen with respect to the natural orifice,
wherein said generally closed body is adapted to be compressed during deployment through the natural orifice and expanded to at least the size of the portion of the lumen upon completion of deployment.

33. The device as claimed in claim 32 wherein said generally closed body is made substantially from a self-expanding material.

34. The device as claimed in claim 32 wherein said generally closed body is configured to be deployed transorally and to be retained at the pylorus, wherein said body generally blocks passage of gas used to insufflate the stomach from distending the bowel.

35. The device as claimed in claim 32 wherein said generally closed body is configured to be deployed transorally and to be retained at the stoma of a gastric pouch, wherein said body blocks passage of gas used to insufflate the pouch from distending the bowel.

36. The device as claimed in claim 32 wherein said generally closed body comprises a diaphragm and at least one retention flange generally surrounding said diaphragm, said retention flange adapted to retain said generally closed body at the portion of the lumen.

37. The device as claimed in claim 36 wherein said diaphragm is generally impenetrable.

38. The device as claimed in claim 36 wherein said diaphragm is penetrable.

39. The device as claimed in claim 32 in combination with another body having a size and shape to be supported in an incision in the luminal wall, wherein said another body is penetrable to pass instruments through the incision while protecting the wall of the lumen at the incision from abrasion resulting from the passage of instruments, and wherein said another body is adapted to substantially seal the luminal wall at the incision against the exchange of fluid between the lumen and the space adjacent to the lumen.

40. A method of performing a natural orifice luminal procedure, comprising:
providing a generally closed body, said generally closed body having a size and shape to be retained by a portion of the lumen defining an aperture between the lumen and a space adjacent to the lumen;
retaining said generally closed body in the aperture between the lumen and a space adjacent to the lumen; and
substantially sealing the aperture from passage of gas between the lumen and the space adjacent to the lumen with said body and maintaining the lumen and the space adjacent to the lumen at different gas pressures.
